(19) United States
(12) Patent Application Publication
(10) Pub. No.: US 2018/0110633 A1
(43) Pub. Date: Apr. 26, 2018

(54) Volume Adjustable Full-Suspension Socket

(71) Applicant: Shaun O'Brien, Indianapolis, IN (US)

(72) Inventor: Shaun O'Brien, Indianapolis, IN (US)

(73) Assignee: Shaun O'Brien, Indianapolis, IN (US)

(21) Appl. No.: 15/332,856

(22) Filed: Oct. 24, 2016

(51) Int. Cl. A61F 2/80; A61F 2/78

(52) U.S. Cl. CPC A61F 2/80; A61F 2002/5035; A61F 2/7812

(57) ABSTRACT

The Prosthetic industry has never had a design that adequately addresses the Above Knee (AK) chronic patient issue of daily volume loss, or the issue of socket rotation, or reliable suspension. Below Knee (BK) amputees can ambulate freely and safely with a variety of suspension technologies. This is due to the fact that the BK amputee does not have excessive soft tissue, resulting in volume loss and limb elongation. Also, the underlying BK boney anatomy prevents rotation. The AK limb is comprised mostly of soft tissue, therefore, until now; all socket control has inadequately come from a variety of brim shapes and styles. The Volume Adjustable Full-Suspension Socket solves all of these issues for the AK amputee and allows them the same security and freedoms as their BK counterparts.

### SUMMARY

This patent covers a volume adjustable above knee socket utilizing a sliding lateral wall. This new approach to above knee prosthetic socket design does address the patient problem of residual limb volume loss or gain, but it, also, provides an improved suspension technique and eliminates the chronic above knee prosthetic socket issue of prosthetic rotation.

### BACKGROUND

Formally, above knee sockets have been suspended distally or through suction. Both of these systems do not accommodate volume fluctuations in the residual limb and do not address the issue of socket ration. The result is a patient's lost confidence in the suspension of their prosthetic limb. This adversely impacts the patient's ability to complete activities of daily life, as well as, participating in hobbies and sports.

This new system is one that addresses suspension in lieu of any daily volume fluctuations that a patient may experience. It is an improved suspension system over what is currently in the market. Also, it simultaneously addresses socket rotation, which is an issue with which all above knee patients struggle. Prior designs have relied heavily on ischial tuberosity containment within the socket to address rotation. This results in a rigid sectional of the wall protruding uncomfortably into the patient's sensitive perineum or buttocks. With the design outlined in this patent, there is not as much of a reliance on ischial containment to control socket rotation and, thus, there is a significant increase in patient comfort. Also, this system controls rotation in a more effective manner than what ischial containment provides; including rotational control during the swing phase of the gait cycle.

### DETAILED DESCRIPTION OF INVENTION

This utility patent covers the volume adjustability of an AK socket. Until now, this presented an impossible hurdle for patients and clinical practitioners alike. Patients who gained or lost more than 10 lbs. would have to be refitted for a new socket at the expense of the taxpayer or insurance company. Also, volume adjustments throughout the day leave patients frequently with an internally rotating leg since the design of the socket set at the initial patient volume; usually casting measurement is performed during a morning evaluation. The patient loses volume throughout the day and the anatomy no longer fits appropriately within the socket to control leg rotation and provide adequate stability. Suction sockets completely lose their suspension properties when the residual limb's fluids are pushed out and volume is lost by day's end. All other distal and proximal liner suspension methods require the patient to take off their leg throughout the day and add socks to fill the void, keeping the patient from dropping into their socket and crashing their sensitive anatomy against the hard brim of the AK (Above Knee) socket. Socks cause bunching, are uncomfortable, and can only accommodate up to a 10 ply fluctuation of volume.

The VAFS Socket is incredibly comfortable and can use the whole lateral and medial internal walls of the AK socket for suspension because there are no socks that need to be added that would nullify the Velcro's ability to suspend the leg. The gel liner that most AK patients don prior to putting on a leg is suspended through suction and adhesion to the dermal layer. These liners are available in a loop fabric cover. There has never been a method to keep the hook counterpart in firm contact with this cover allowing for the most intimate and comfortable suspension method available today. As the volume decreases throughout the day, the patient simply tightens the two lateral ratchet systems that attach anteriorly and posteriorly (4 in total) and the lateral wall uniformly tightens the socket along the frontal plane keeping the medial and lateral Velcro firmly positioned against the loop AK gel liner fabric cover. The patient can adjust suspension and volume without ever having to take off their limb. This means that they can confidently engage in high activity sports or activities where sweat and volume loss quickly become critical failure risks. There is a distal flexible lateral wall fixation strap that keeps that lateral wall mobile but inhibits it from migrating anteriorly and compromising suspension.

The socket can be made of any common materials used in the industry currently. It can be a completely hard socket composed of fiberglass, carbon, and acrylic resin. It can have this hard socket and a flexible Proflex inner liner, which allows for cutouts and a more flexible socket. In this case, the lateral wall fits between the flexible inner socket and the external frame. Thus, there is no risk for impingement on the tissue when the socket is tightened. The skin is protected by the liner. The lateral wall needs to be flexible and is recommended to be made of polyethylene, but any flexible plastic will suffice. Hard sockets are the norm in the Prosthetic industry, but this system can work on plastic sockets too, such as polypropylene or copolymer plastics (a combination of polypropylene and polyethylene). Lastly, the socket is donned with the lateral wall disengaged/open. Once the leg is positioned the way that is most comfortable for the patient, the individual closes the lateral wall bringing up the anterior edge and sliding it inside the socket (shown by the overlap in the drawings). This puts pressure on the medial hook Velcro and engages the lateral hook system, as well. The leg is now suspended and the patient continues to tighten until there is adequate confidence in the suspension and a slight proximal migration of the residual limb tissue that provides a comfortable barrier for the ischial tuberosity within the socket walls and the ramus above the walls of the socket.

### BRIEF DESCRIPTION OF THE DRAWINGS

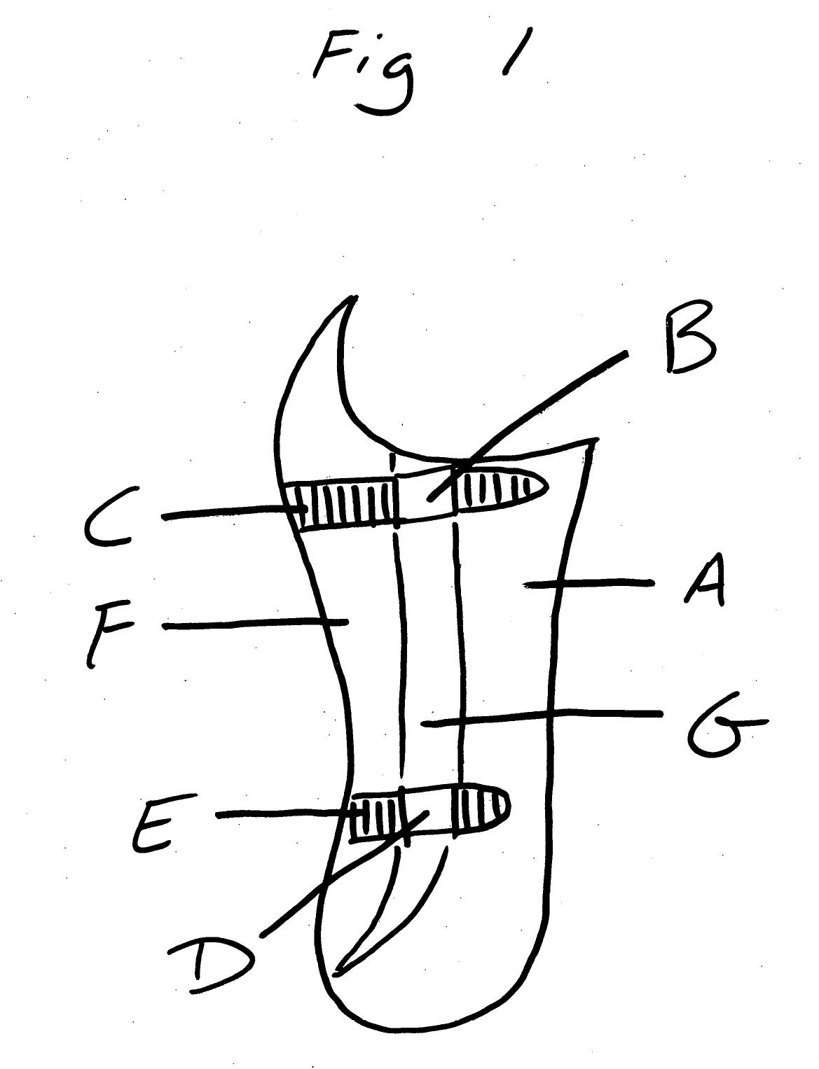

### FIG. 1

### Anterior View

A is the medial panel of the above knee socket, this panel can be constructed of a variety of materials to include copolymer (polypropylene and polyethylene) plastic, polypropylene, acrylic resin and fiberglass, acrylic resin and carbon fiber, and can include a flexible inner liner, such as Proflex, if the clinician feels that would be most appropriate for the above knee amputated patient. This panel will comprise the majority of the socket's structure. B is one of two plastic ratchets that is used to tighten the lateral panel, with a hook Velcro strip included, against the patient's loop based inner liner. C is the grooved plastic strap that fits with the ratchet. The ratchet and straps are not proprietary items to this patent and can be bought from a variety of sources on the open market. D is another identical ratchet. E is another plastic grooved strap. F is the lateral panel with another strip of hook Velcro included and is the portion of the socket that flexes and tightens to the patient's limb. Because of this flexibility, it is recommended that this portion of the socket is made out of polyethylene. G is the overlapping portion of the socket where the thin, ⅛ inch, polyethylene F slides inside the rigid G above knee socket portion. If a flexible inner liner is utilized, F will slide in-between the inner flexible liner and the rigid G above knee socket.

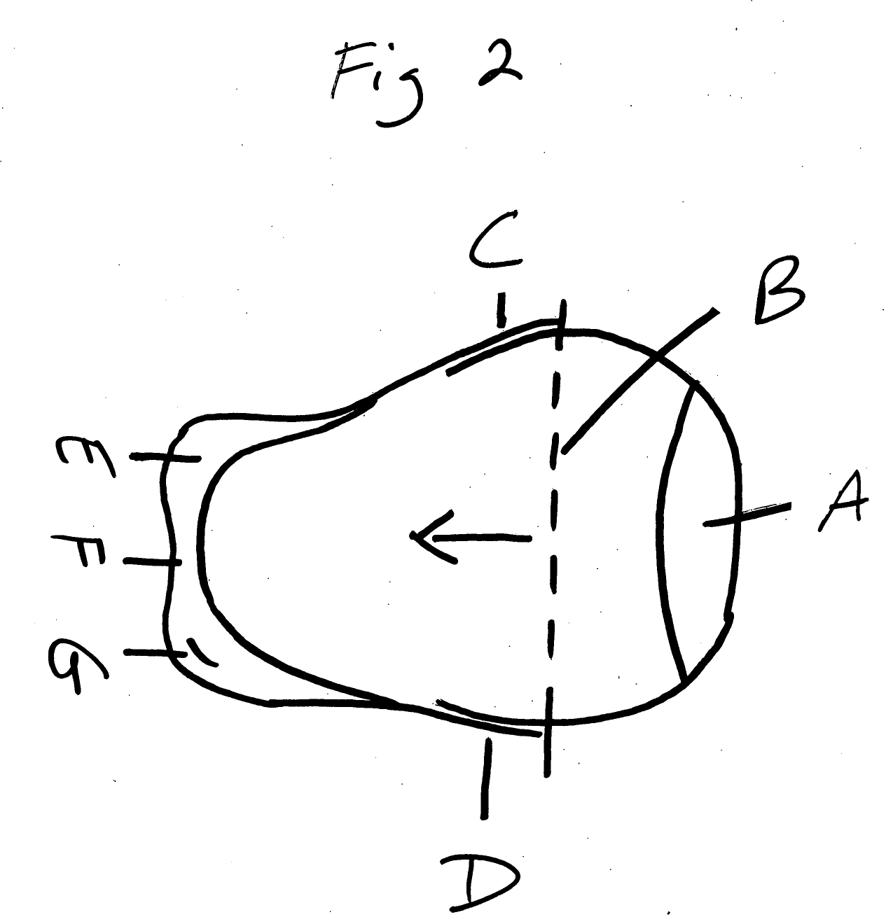

### FIG. 2

### Superior View

A is representative of the proximal cupping portion of the above knee socket. This is not proprietary to this patent and is commonly utilized in the above knee socket design. B is the plane that demarcates that area where the lateral portion of the socket slides inside the rigid medial socket. C and D show the overlapping of the two panels, medial and lateral. E is the relief for the adductor tendon and F is the relief for the ramus and G is the ischial seat, these are all common parts of an above knee socket brim design and are not propriety to this patent. This patent can be utilized with any socket shape and focuses on improved above knee suspension, eliminating rotation, and volume control. These are all areas of chronic concern in the industry today.

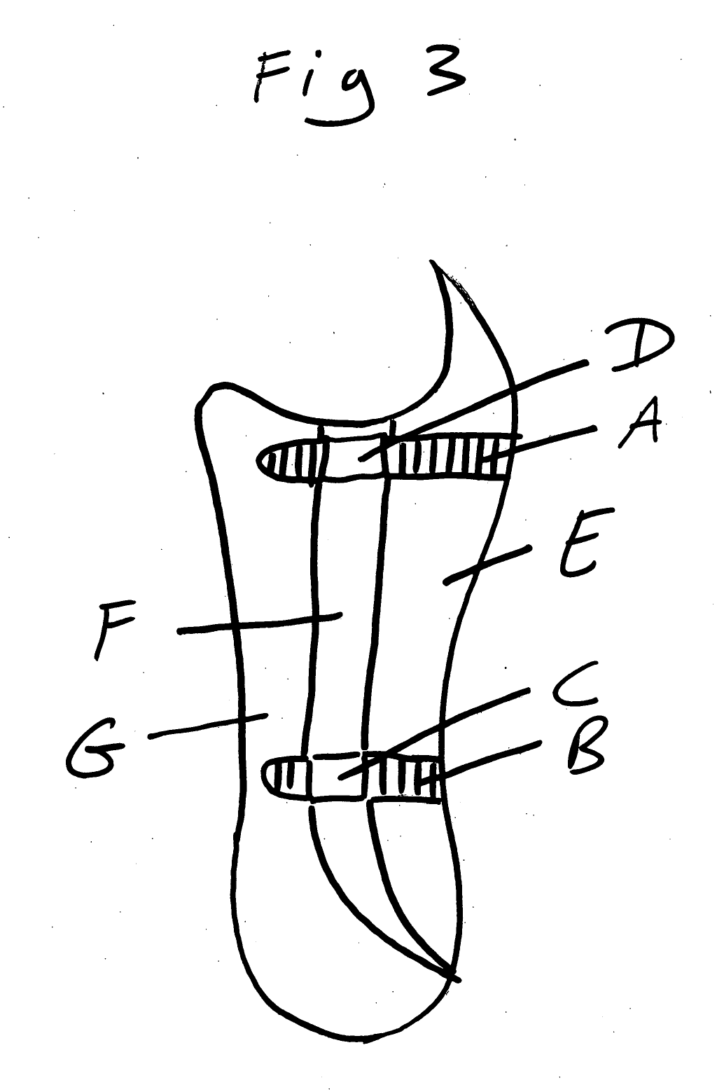

### FIG. 3

### Posterior View

This view provides no new insight into this new design. It is provided to complete the visual image of the concept. A and B are the grooved straps. C and D are the ratchets. E is the lateral portion of the above knee socket. F is the medial lateral overlap. G is the medial above knee panel.

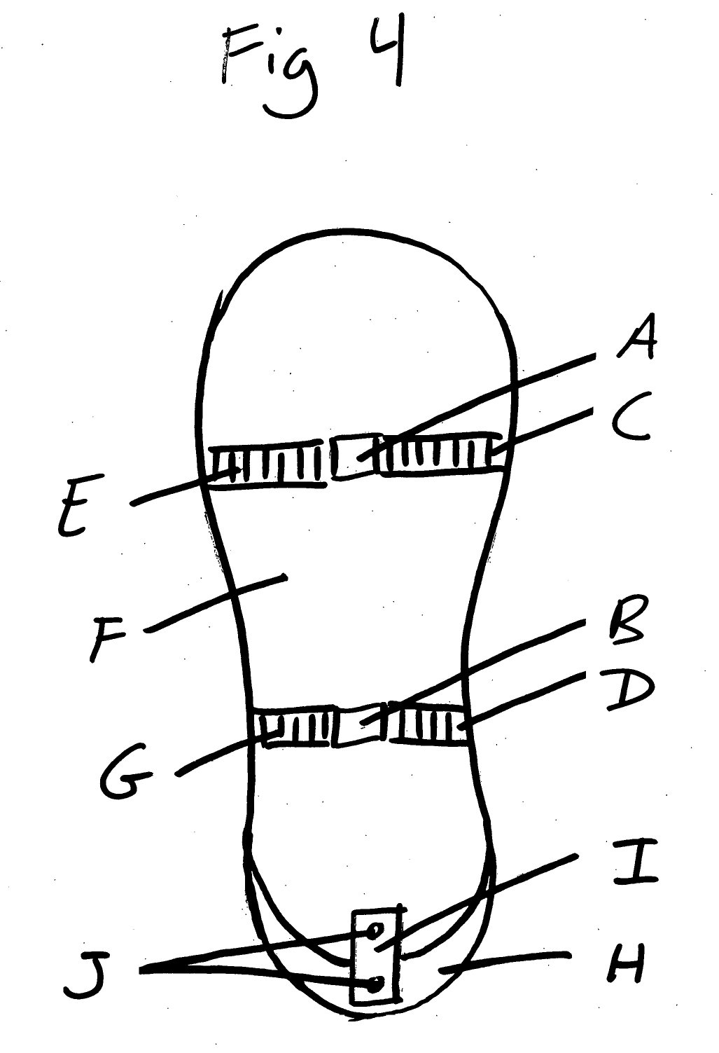

### FIG. 4

### Lateral View

This is an important perspective because it denotes a critical component of the design: I and J. I is a strap that can be constructed of any material but it is recommended that it is made of Dacron webbing. J is the attachment of the Dacron strap to the two sockets: medial H and lateral F. J are rivets or screws. I prevents the distal drift of the medial panel H as the patient enters the swing phase of the gait cycle, human walking. Without this strap, suspension of the lower half of the above knee prosthesis, including the knee, shin, ankle, and foot, would become a problem. I prevents the prosthesis from pulling away from the amputee and ensures an intimate and secure fit. A and B are ratchets. C, D, E, and G are grooved straps.

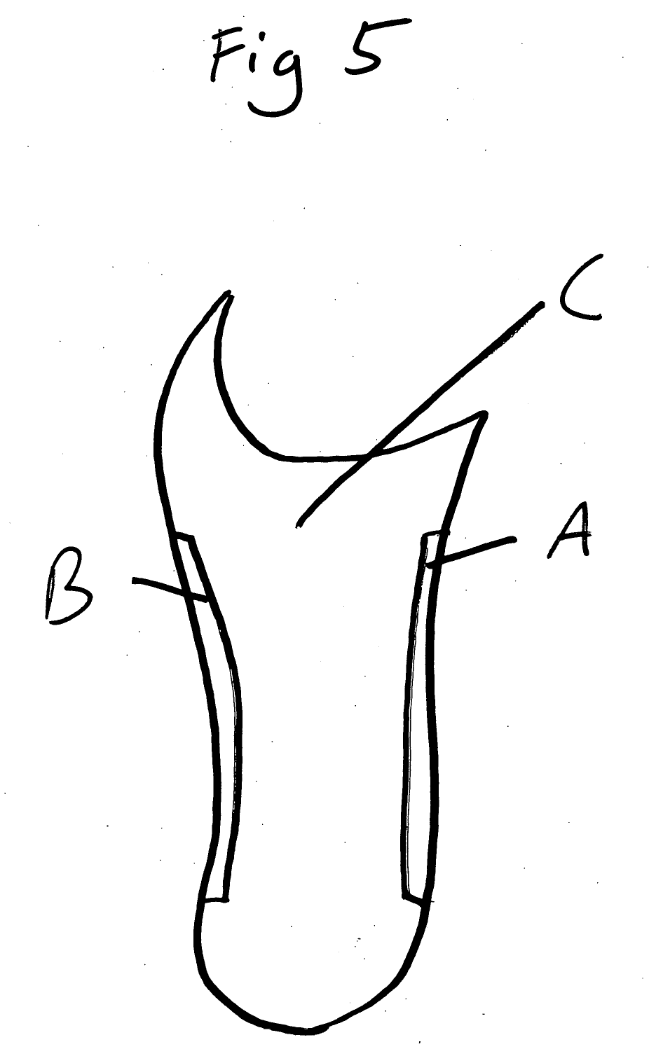

### FIG. 5

### Anterior View Suspension

A and B represent the hook Velcro that are glued to the inside of the lateral and medial panels of C. This is the critical component of suspension in this design. They can be made of any size or shape Velcro strips or pads but it is recommended that they be made of 1″, 1½″, or 2″ wide hook Velcro strips. By tightening the ratchet system, the lateral panel moves medially and presses both hook strips, medial and lateral, firmly against the patient's loop fabric covered gel or silicone inner liner, of which there are multiple versions currently available on the open market. The gel or silicone liner adheres to the patient's skin and the VAFS socket system eliminate pistoning between the gel or silicone liner and the socket, creating the most intimate prosthetic socket fit available today.

1. An above knee prosthetic socket that utilizes Velcro pressed against an inner gel or silicone liner for suspension and which eliminates the tendency for socket rotation.

2. An above knee socket that creates the force necessary to keep the prosthesis suspended, via Velcro, by utilizing an adjustable sliding lateral wall.

3. A lateral above knee socket wall that slides inside of the above knee socket and is attached anteriorly and posteriorly with any means necessary to adjust the lateral wall with the patient desired level of inward socket pressure.

4. A above knee socket that achieves volume adjustments, accommodating the fluctuating fluid accumulations or reductions of the amputated residual limb, through the use of an adjustable lateral wall.
